(19) United States
(12) Patent Application Publication
(10) Pub. No.: US 2010/0098752 A1
(43) Pub. Date: Apr. 22, 2010

(54) Materials and Methods for Delivering Antioxidants into the Skin

(76) Inventor: Mark A. Pinsky, Palm Beach Gardens,, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/523,237

(22) PCT Filed: Jan. 18, 2008
(86) PCT No.: PCT/US08/51437

§ 371 Date: Dec. 3, 2009

Related U.S. Application Data
(60) Provisional application No. 60/885,643, filed on Jan. 19, 2007.
(60) Provisional application No. 60/885,641, filed on Jan. 19, 2007.
(60) Provisional application No. 60/885,551, filed on Jan. 18, 2007.
(60) Provisional application No. 60/885,549, filed on Jan. 18, 2007.
(60) Provisional application No. 60/885,523, filed on Jan. 18, 2007.
(60) Provisional application No. 60/885,482, filed on Jan. 18, 2007.

(51) Int. Cl. A61K 9/127; A61K 8/14; A61K 38/02; A61K 31/34; A61K 31/355; A61K 38/43; A61K 31/12; A61K 36/82; A61K 36/87; A61K 31/315; A61K 36/42; A61K 36/06; A61K 38/08; A61P 43/00

(52) U.S. Cl. 424/450; 424/59; 514/2; 514/474; 514/458; 424/94.1; 514/690; 424/729; 424/766; 514/494; 424/758; 424/195.15; 514/17

(57) ABSTRACT

Compositions and methods for administering one or more antioxidants to a human subject have been developed. The antioxidant-containing lipid vesicles of the invention provide a delivery system for antioxidants which can be applied topically to the skin.

### BACKGROUND OF THE INVENTION

The skin is the largest organ in the human body and consists essentially of two primary layers—the epidermis and the dermis. The epidermis is the outermost layer and, among other things, controls water loss from cells and tissue. The dermis is the layer below the epidermis and contains blood vessels, lymph vessels, hair follicles and sweat glands. Below the dermis is the hypodermis. Although the hypodermis is considered to be part of the integumentary system, it is not generally considered to be a layer of the skin. The hypodermis is used mainly for fat storage.

The outermost epidermis is made up of stratified squamous epithelium with an underlying basement membrane. It contains no blood vessels, and is nourished by diffusion from the dermis. The main type of cells that make up the epidermis are keratinocytes, with melanocytes and Langerhans cells also present. The epidermis can be further subdivided into the following strata (beginning with the outermost layer): corneum, lucidum, granulosum, spinosum, basale. Cells are formed through mitosis at the innermost layers. They move up the strata changing shape and composition as they differentiate and become filled with keratin. They eventually reach the corneum and become sloughed off. This process is called keratinization and takes place within about 30 days.

Various means for delivery of substances to or into the skin have been proposed.

U.S. Pat. No. 5,354,564 discloses personal care products comprising an aqueous dispersion of particles of silicone wherein said particles have a surface modifier adsorbed on the surface thereof in an amount sufficient to achieve a particle size of less than about 400 nanometers (nm).

U.S. Pat. No. 5,660,839 discloses incorporating deformable hollow particles into cosmetic and/or dermatological compositions containing fatty substances, to markedly reduce or eliminate the sticky and/or greasy feel attributed to these fatty substances.

U.S. Pat. No. 5,667,800 discloses an aqueous suspension of solid lipoid nanoparticles, comprising at least one lipid and preferably also at least one emulsifier, for topical application to the body.

U.S. Pat. No. 5,780,060 discloses microcapsules with a wall of crosslinked plant polyphenols and compositions containing them. The microcapsules are obtained by the interfacial crosslinking of plant polyphenols, particularly flavonoids.

U.S. Pat. Nos. 5,851,517 and 5,945,095 disclose compositions including a dispersion of polymer particles in a non-aqueous medium. A dispersion of surface-stabilized polymer particles can be used in a non-aqueous medium, in a cosmetic, hygiene or pharmaceutical composition. The dispersions may, in particular, be in the form of nano-particles of polymers in stable dispersion in a non-aqueous medium.

U.S. Pat. Nos. 5,759,526 and 5,919,487 disclose nanoparticles coated with a lamellar phase based on silicone surfactant and compositions containing them. The nanoparticles, and in particular nanocapsules, provided with a lamellar coating obtained from a silicone surfactant, can be used in a composition, in particular a topical composition, for treatment of the skin, mucosae, nails, scalp and/or hair.

U.S. Pat. No. 5,188,837 discloses a microsuspension system and method for its preparation. The microsuspension contains lipospheres which are solid, water-insoluble microparticles that have a layer of a phospholipid embedded on their surface. The core of the liposphere is a solid substance to be delivered or a substance to be delivered that is dispersed in an inert solid vehicle such as a wax.

U.S. Pat. No. 4,919,841 discloses a process for preparing encapsulated active particles by the steps of: dispersing active materials in molten wax; emulsifying the active/wax dispersion in an aqueous surfactant solution for no longer than 4 minutes; quenching the capsules by cooling; and retrieving solidified capsules. Examples of active materials are fragrances.

Each of these methods has disadvantages.

Liposomes are vesicular lipid membrane structures that enclose, for example, a volume of water. The existence of liposomes has been known for many years. In the early 1900's, researchers, studying isolated lecithin (phosphatidylcholine), cephalin (phosphatidylethanolamine/phosphatidylserine), phrenosin (galactosyl ceramide) and kerasin (glucosyl ceramide), found that all of these molecules would swell in water to form hydrated multilamellar layers, consisting of lipid bilayers separated by water. Also, mixtures of ionic and nonionic lipids dispersed in water were found to form stable “emulsions” in which the lipid molecules take up positions side by side to form a homogeneous mixed phase. These emulsions were the equivalents of what are now called multilamellar liposomes.

Physical and chemical studies have shown that amphiphiles form certain preferred arrays in the presence of water. Formation of these arrays, which include micelles, monolayers and bimolecular layers, is driven by the need for the polar head groups, which may be ionogenic or not, to associate with water and the need of the apolar, hydrophobic tail to be excluded from water. Exactly which type of structure is assumed depends upon the nature of the amphiphile, its concentration, the presence of other amphiphiles, temperature, and presence of salt and other solutes in the aqueous phase.

Until recently, liposome technology has been concerned mostly with vesicles composed of phospholipids, predominantly phosphatidylcholine, and these continue to be the focus of most publications and patents. However, although phospholipids are suitable for certain pharmaceutical applications, phospholipid liposome technology has been beset by serious problems, for example, phospholipids turn over rapidly in vivo and are unstable in storage. Also, they are labile and expensive to purify or synthesize, and the manufacture of phospholipid liposomes is difficult and costly to scale up.

### BRIEF SUMMARY OF THE INVENTION

The subject invention pertains to new and advantageous skin care compositions. In a preferred embodiment, the subject invention provides lipid vesicles (liposomes) incorporating at least one agent selected from antioxidants, anti-inflammatory agents, peptides, humectants, sunscreen agents, and emollients.

Antioxidants are enzymes or other organic molecules that counteract the damaging effects of oxidative free radical molecules in cells or tissues by safely reacting with these free radicals. When applied to the skin according to the subject invention antioxidants provide protection against the damaging effects of UV radiation and free radicals.

Anti-inflammatory agents are substances that provide pain relief and reduce inflammation and typically provide pain relief. When applied to the skin according to the subject invention, anti-inflammatory agents can deliver such pain relief and inflammation-reducing effects directly to the skin.

Peptides are molecules made up of amino acids and can combat signs of aging in skin. When applied to the skin according to the subject invention, peptides provide the skin with increased density and increased ability to produce collagen to support the skin. Peptides can also improve fibronectin synthesis and cell adhesion structural integrity, enhance dermal repair mechanisms and the immune response of the skin, facilitate the elimination of pigments responsible for dark circles, and reduce the puffiness and bags under the eyes.

A humectant is a substance with the ability to attract water molecules. When applied to the skin according to the subject invention, humectants provide protection against dry skin and wrinkles.

Sunscreen agents are organic or inorganic compounds that counteract the damaging effects of ultraviolet radiation by reflecting, scattering, and/or absorbing such radiation. When applied to the skin according to the subject invention, sunscreen agents provide protection against ultraviolet radiation.

Emollients are substances which soften and soothe the skin. When applied to the skin according to the subject invention, emollients provide protection against dry skin.

Particularly preferred antioxidants are L-ascorbic acid, Vitamin E (tocopherol), tocopheryl acetate, coenzyme Q-10, white tea extract, grape seed extract, niacinamide, and/or zinc citrate.

Particularly preferred anti-inflammatory agents are cucumber extract, ivy extract, shitake extract, and/or allantoin. A

Particularly preferred peptides are hexapeptide-3 (Argireline), hexapeptide-9 (Collaxyl), Dermaxyl™ (palmitoyl oligopeptide), Matrixyl 3000™ (glycerin, butylene glycol, water, carbomer, polysorbate-20, palmitoyl oligopeptide, and palmitoyl tetrapeptide-3), Haloxyl™ (palmitoyl tetrapeptide-3), Sepilift™ (dipalmitoyl hydroxyproline), Eyeliss™ (hesperidin methyl chalcone and dipeptide-2 with palmitoyl tetrapeptide-3), Rigin, and/or Maxilip™ (ethylhexyl palmitate, tribehenin, sorbitan isostearate, and palmitoyl oligopeptide).

Particularly preferred humectants are avocado oil/sterol, avocado butter, white petrolatum, and/or illipe butter.

Particularly preferred sunscreen agents are octocrylene, zinc oxide, and/or octyl methoxycinnannate.

Particularly preferred emollients are of illipe butter, shea butter, shora seed butter, Ceraphyl 847® (octyldodecyl stearoyl stearate), C12-15 alkyl benzoate, pentaerythrityl tetraisostearate, and/or diisopropyl adipate.

The compositions may also contain additional skin care agents.

The subject invention further pertains to methods of using such lipid vesicles for delivery of active ingredients to a patient to achieve enhanced skin care.

In one embodiment, the present invention provides a skin care composition with one or more active agents, wherein the formulation facilitates the active ingredients passing through the epidermis and thus being released within the dermis of the skin. In a further embodiment, the agent may be delivered to the epidermis as well. Accordingly, the present invention is useful in regulating and/or improving the condition of the skin (including the appearance and/or feel of the skin) by efficiently delivering an antioxidant to the appropriate location within the skin.

The present invention also relates to methods of using such compositions to regulate and/or improve the condition of skin. The methods of the subject invention generally include the step of topically applying the compositions to the skin (epidermis) of the patient needing such treatment, wherein a therapeutically effective amount of such composition is applied.

Advantageously, the present invention provides compositions and methods for combating the aging of skin, wherein combating the aging of skin can include, for example, hydration of the skin, treating the appearance of wrinkles, fine lines, and other forms of undesirable skin texture. By presenting active agents into the dermal and/or epidermal layer(s) of the skin, the form, strength, as well as function of the skin is enhanced.

In certain embodiments, the compositions of the subject invention comprise a dispersion of lipid vesicles that contain multiple agents that are useful in delaying, minimizing, or eliminating skin aging, wrinkling, and/or other histological changes typically associated with the intrinsic conditions (such as aging, menopause, acne, etc.) and extrinsic conditions (such as environmental pollution, wind, heat, low humidity, harsh surfactants, etc.).

In an exemplary embodiment of the invention, non-phospholipid paucilamellar lipid vesicles incorporating at least one active agent are used to deliver the active agent(s) into the skin of a human subject. Non-phospholipid paucilamellar lipid vesicles are particularly advantageous for use in the invention as such vesicles are stable and inexpensive to manufacture, and also feature a large cavity size for holding an active ingredient. In an alternative embodiment, cyclodextrins are used to deliver the active agents to the dermis layer of the skin.

### DETAILED DESCRIPTION

The present invention is directed to materials and methods for the topical administration of a therapeutically effective amount of one or more active agents to a specific layer within the skin in order to improve the condition of the skin. Accordingly, in a preferred embodiment, the present invention provides compositions, and methods for using such compositions, comprising a dispersion of lipid vesicles that contain at least one active agent, wherein the lipid vesicles facilitate penetration through the epidermis and dispersal of the vesicle contents, into the dermis layer of the skin.

Improvement of skin condition is often desired due to conditions that may be induced or caused by factors internal and/or external to the body. Examples include, but are not limited to, environmental damage, smoking, radiation exposure (including ultraviolet radiation), chronological aging, menopausal status (e.g., post-menopausal changes in skin), stress, diseases, etc.

The present invention is useful for therapeutically and/or prophylactically improving visible and/or tactile characteristics in skin. For example, in one embodiment, the length, depth, and/or other dimension of lines and/or wrinkles are decreased and hydration is achieved.

“Improving skin condition” includes prophylactically preventing or therapeutically treating a skin condition, and may involve one or more of the following benefits: thickening of skin, preventing loss of skin elasticity, and a reduction in lines or winkles.

Following are additional definitions relevant to the subject invention. It should be appreciated that the following definitions are used throughout this application. Unless otherwise defined, all technical terms used herein have the same meaning as commonly understood by one or ordinary skill in the art to which this invention belongs.

The term “epidermis” or “epidermal,” as used herein, refers to the outermost layer of the skin.

The term “topical application,” as used herein, means to apply or spread the compositions of the present invention onto the surface of the epidermis tissue.

The term “dermatologically-acceptable,” as used herein, means that the compositions or components thereof so described are suitable for use in contact with mammalian epidermal tissue without undue toxicity, incompatibility, instability, allergic response, and the like.

The term “therapeutically effective amount,” as used herein, refers to an amount of a compound (such as an antioxidant, anti-inflammatory agent, peptide, humectant, sunscreen agent, or emollient) or composition sufficient to induce a positive benefit, preferably a positive skin appearance and/or feel. In accordance with the subject invention, the therapeutically effective amount is an amount of an active agent, either alone or in combination with other agents, that regulates and/or improves the skin, but where the amount is low enough to avoid serious side effects, i.e., to provide a reasonable benefit to risk ratio, within the scope of sound judgment of the skilled artisan.

The term “sagging” as used herein means the laxity, slackness, or the like condition of skin that occurs as a result of loss of, damage to, alterations to, and/or abnormalities in dermal structure and/or function.

The terms “smoothing” and “softening,” as used herein, refer to altering the surface of the epidermis tissue such that its tactile feel is improved.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity, sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

As used herein, “shear mixing” means the mixing of a lipophilic phase with an aqueous phase under turbulent or shear conditions that provide adequate mixing to hydrate the lipid and form lipid vesicles

By the terms “disperse” and “dispersion” are meant dissolution or forming a suspension or colloid to yield a flowable phase.

As used herein, a “nucleic acid” or a “nucleic acid molecule” means a chain of two or more nucleotides such as RNA (ribonucleic acid) and DNA (deoxyribonucleic acid). A “recombinant” nucleic acid molecule is one made by an artificial combination of two otherwise separated segments of sequence, e.g., by chemical synthesis or by the manipulation of isolated segments of nucleic acids by genetic engineering techniques.

The terms “protein” and “polypeptide” are used synonymously to mean any peptide-linked chain of amino acids, regardless of length or post-translational modification, e.g., glycosylation or phosphorylation. A “purified” polypeptide is one that has been substantially separated or isolated away from other polypeptides in a cell or organism in which the polypeptide naturally occurs (e.g., 90, 95, 98, 99, 100% free of contaminants).

When referring to a nucleic acid or polypeptide, the term “native” refers to a naturally-occurring nucleic acid or polypeptide.

The compositions of the present invention, which enable dermal layer dispersion of the active ingredient, are useful for improving the skin, including improving skin appearance and/or feel. For example, compositions of the present invention are useful for improving the appearance of skin condition by providing a visual improvement in skin appearance following application of the composition to the skin.

Advantageously, the compositions of the present invention may have additional desirable properties, including stability, long shelf-life, absence of significant skin irritation, and good aesthetics. In certain embodiments, to accomplish such additional benefits, the compositions of the invention further comprise agents, in addition to the antioxidant, that promote composition stability, reduce skin irritation, and/or enhance the aesthetic appeal of the composition.

Examples of good aesthetics include compositions, such as luxurious creams and lotions, that (i) are light and nongreasy, (ii) have a smooth, silky feel upon the skin, (iii) spread easily, and/or (iv) absorb quickly. Other examples of good aesthetics include compositions that have a consumer acceptable appearance (i.e. no unpleasant odor or discoloration present), and provide good skin feel.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the particular embodiments discussed below are illustrative only and not intended to be limiting.

### Antioxidants

Antioxidants are enzymes or other organic molecules that counteract the damaging effects of oxidative free radical molecules in cells or tissues by safely reacting with these free radicals.

Antioxidants are especially important in the mitochondria of eukaryotic cells since the use of oxygen as part of the process for generating energy produces reactive oxygen species. The process of aerobic metabolism requires oxygen because it serves as the final resting place for electrons generated by the oxidation steps of the citric acid cycle.

Additionally, research suggests that antioxidants reduce damage to cells and biochemicals from free radicals. This may slow down, prevent, or even reverse certain diseases that result from cellular damage, and perhaps even slow down the natural aging process. Some antioxidants, such as Vitamin E, preserve, or even recycle, other antioxidants.

Human skin is equipped with a network of enzymatic and nonenzymatic antioxidant defense systems, including tocopherols, ascorbate, polyphenols, and carotenoids. However, when these compounds or other antioxidants are administered, they provide an additional protective effect on the skin and skin cells. Antioxidants applied topically may play an important role in counteracting the oxidative injury to lipids, proteins, and hydrophilic molecules on the skin and eye that are provoked by radical oxygen species.

Moreover, antioxidants provide protection against UV radiation which can cause increased scaling or texture changes in the stratum corneum and against other environmental agents which can cause skin damage.

In a preferred embodiment of the subject invention, one or more antioxidants are incorporated into lipid vesicles in order to administer antioxidants to the skin of a patient. As described herein, any lipid vesicle suitable for encapsulating one or more antioxidants for administering to the skin of a human subject may be used.

Examples of antioxidants that may be used according to the subject invention are ascorbic acid (Vitamin C) and its salts, L-ascorbic acid, ascorbyl esters of fatty acids, ascorbic acid derivatives (e.g., magnesium ascorbyl phosphate, sodium ascorbyl phosphate, ascorbyl sorbate), tocopherol (Vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts (such as white tea extract), lycopene, grape seed extract, grape skin extract, melanin, Coenzyme Q-10, niacinamide, zinc citrate, and rosemary extracts.

Particularly preferred is the use of L-ascorbic acid, Vitamin E (tocopherol), tocopheryl acetate, coenzyme Q-10, white tea extract, grape seed extract, niacinamide, and/or zinc citrate.

Vitamin E protects cell membranes from peroxidation and scavenges free radicals. Coenzyme Q-10 inhibits lipid peroxidation in plasma membranes, functions as a coenzyme in the energy-producing adenosine triphosphate pathways found in the mitochondria of every cell in the body, and may have some efficacy in preventing the detrimental effects of ultraviolet radiation exposure. Grape seed extract enhances vision, protects the skin against UVB damage, fosters wound healing, and may be a more potent scavenger of free radicals than are Vitamins C and E. Niacinamide increases synthesis of collagen and lipids, inhibits the transfer of melanosomes, and decreases inflammation. Niacinamide also increases biosynthesis of ceramides as well as other intercellular lipids in the stratum corneum.

Anti-Inflammatory Agents

Anti-inflammatory agents are substances that help relieve pain as well as reduce inflammation. They are classified as steroidal anti-inflammatory agents and non-steroidal anti-inflammatory agents.

Steroidal anti-inflammatory agents reduce inflammation by binding to cortisol receptors. Non-steroidal anti-inflammatory agents alleviate pain by counteracting the cyclooxygenase (COX) enzyme and reduce inflammation by preventing synthesis of prostaglandins.

Most non-steroidal anti-inflammatory agents act as non-selective inhibitors of the enzyme cyclooxygenase, inhibiting both the cyclooxygenase-1 (COX-1) and cyclooxygenase-2 (COX-2) isoenzymes. Cyclooxygenase catalyses the formation of prostaglandins and thromboxane from arachidonic acid (itself derived from the cellular phospholipid bilayer by phospholipase A₂). Prostaglandins act, among other things, as messenger molecules in the process of inflammation.

Research suggests that prostaglandins are mediators of inflammation in the skin and that prostaglandins are synthesized locally in response to the inflammatory stimulus.

Additionally, the anti-inflammatory substances enhance the skin appearance of a subject by contributing to a more uniform and acceptable skin tone or color.

In a preferred embodiment of the subject invention, one or more anti-inflammatory agents are incorporated into lipid vesicles in order to administer anti-inflammatory agents to the skin of a patient. As described herein, any lipid vesicle suitable for encapsulating one or more anti-inflammatory agents for administering to the skin of a human subject may be used.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

Some non-steroidal anti-inflammatory agents useful in the compositions of the subject invention include, but are not limited to:

1) the oxicams, such as piroxicam, isoxicam, tenoxicam, sudoxicam, and CP-14,304;

2) the salicylates, such as aspirin, disalcid, benorylate, trilisate, safapryn, solprin, diflunisal, and fendosal;

3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;

4) the fenamates, such as mefenamic, meclofenamic, flufenamic, niflumic, and tolfenamic acids;

5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and

6) the pyrazoles, such as phenylbutazone, oxyphenbutazone, feprazone, azapropazone, and trimethazone.

For a detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Mixtures of these non-steroidal anti-inflammatory agents may also be employed, as well as the dermatologically acceptable salts and esters of these agents. For example, etofenamate, a flufenamic acid derivative, is particularly useful for topical application. Of the nonsteroidal anti-inflammatory agents, ibuprofen, naproxen, flufenamic acid, etofenamate, aspirin, mefenamic acid, meclofenamic acid, piroxicam and felbinac are preferred; ibuprofen, naproxen, ketoprofen, etofenamate, aspirin and flufenamic acid are more preferred.

Finally, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, sea whip extract, cucumber extract, ivy extract, shitake extract, and allantoin may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C.sub.2-C.sub.24 saturated or unsaturated esters of the acids, preferably C.sub.10-C.sub.24, more preferably C.sub.16-C.sub.24. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Particularly preferred anti-inflammatory agents for use with the present invention are cucumber extract, ivy extract, shitake extract, and allantoin.

### Peptides

Peptides are molecules formed by the linking of amino acids via amide bonds. The amino acids in peptides are also the building blocks of protein.

Certain peptides play an important role in fighting the visible signs of aging in the skin. In a preferred embodiment of the subject invention, one or more peptides are incorporated into lipid vesicles in order to administer peptides to the skin of a patient. As described herein, any lipid vesicle suitable for encapsulating one or more peptides for administering to the skin of a human subject may be used.

Examples of peptides that may be used in a preferred embodiment of the subject invention include hexapeptide-3 (Argireline), hexapeptide-9 (Collaxyl), Dermaxyl™ (palmitoyl oligopeptide), Matrixyl 3000® (glycerin, butylene glycol, water, carbomer, polysorbate-20, palmitoyl oligopeptide, and palmitoyl tetrapeptide-3), Haloxyl™ (palmitoyl tetrapeptide-3), Sepilift™ (dipalmitoyl hydroxyproline), Eyeliss™ (hesperidin methyl chalcone and dipeptide-2 with palmitoyl tetrapeptide-3), Rigin, and Maxilip™ (ethylhexyl palmitate, tribehenin, sorbitan isostearate, and palmitoyl oligopeptide).

Argireline mimics the action of clostridial neurotoxins via catecholamine inhibitors. Collaxyl improves collagen type I synthesis, fibronectin synthesis, and cell adhesion structural integrity of the basement membrane. Dermaxyl™ is a potent chemotactic protein stimulator that enhances dermal repair mechanisms, stimulates fibroblasts, and activates extracellular matrix turnover. Matrixyl 3000™ promotes the synthesis of collagen types I and III and fibronectin by cultured fibroblasts. Haloxyl™ facilitates the elimination of blood-originating pigments responsible for dark circle coloration and inflammation around the eyes. Sepilift™ contracts collagen fibers, stimulates production of procollagen, stimulates inhibitors of MMPs, and significantly reduces superoxide anion. Eyeliss™ reduces the puffiness and bags under the eyes. Rigin mimics DHEA, rejuvenates the immune response of the skin, and restores cytokine (interleukine 6) balance in mature skin. Maxilip™ stimulates collagen and glycosaminoglycan synthesis.

### Humectants

A humectant is a substance with the ability to attract water molecules. It is often a molecule with several hydrophilic groups and the affinity to form hydrogen bonds with water.

Since humectants are hydrophilic, they help retain water. Therefore, when used on the skin, humectants keep the skin moisturized, preventing wrinkles and dry skin in the process.

In a preferred embodiment of the subject invention, one or more humectants are incorporated into lipid vesicles in order to administer humectants to the skin of a patient. As described herein, any lipid vesicle suitable for encapsulating one or more humectants for administering to the skin of a human subject may be used.

Examples of preferred humectants that may be used according to the subject invention are avocado oil/sterol, avocado butter, white petrolatum, and/or illipe butter.

### Sunscreen Agents

Exposure to ultraviolet light can result in excessive scaling and texture changes of the stratum corneum, as well as photooxidative stress including the formation of free radicals and the damage to tissues from those free radicals. Therefore, the compositions of the subject invention may optionally contain a sunscreen agent. As used herein, “sunscreen agent” includes both sunscreen agents and physical sunblocks. Suitable sunscreen agents may be organic or inorganic.

In a preferred embodiment of the subject invention, one or more sunscreen agents are incorporated into lipid vesicles in order to administer sunscreen agents to the skin of a patient. As described herein, any lipid vesicle suitable for encapsulating one or more sunscreen agents for administering to the skin of a human subject may be used.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof.

A wide variety of conventional organic sunscreen agents are suitable for use herein. Sagarin, et al., at Chapter VIII, pages 189 et seq., of Cosmetics Science and Technology (1972), discloses numerous suitable agents. Specific suitable sunscreen agents include, for example: octyl methoxycinnannate, p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butyl methoxydibenzoylmethane; etocrylene; octocrylene; [3-(4′-methylbenzylidene bornan-2-one), terephthalylidene dicamphor sulfonic acid and 4-isopropyl-di-benzoylmethane.

Particularly preferred is the use of octocrylene, zinc oxide, and/or octyl methoxycinnannate.

### Emollients

Emollients are substances which soften and soothe the skin. They are used to correct dryness and scaling of the skin. They are very similar to moisturizers, and in fact, moisturizers are often a collection of emollients.

Moisture is very important in the skin. It helps to fill out the skin and prevents wrinkles and the “dry skin” appearance. Moisture also helps maintain the soft feeling of skin.

In a preferred embodiment of the subject invention, one or more emollients are incorporated into lipid vesicles in order to administer emollients to the skin of a patient. As described herein, any lipid vesicle suitable for encapsulating one or more emollients for administering to the skin of a human subject may be used.

Examples of emollients that may be used according to the subject invention are glycerin, illipe butter, shea butter, shora seed butter, Ceraphyl 847® (octyldodecyl stearoyl stearate), C12-15 alkyl benzoate, pentaerythrityl tetraisostearate, and diisopropyl adipate.

Particularly preferred is the use of illipe butter, shea butter, shora seed butter, Ceraphyl 847® (octyldodecyl stearoyl stearate), C12-15 alkyl benzoate, pentaerythrityl tetraisostearate, and/or diisopropyl adipate

### Lipid Vesicles Containing Active Agents

The invention provides compositions including lipid vesicles incorporating at least one antioxidant. The vesicles containing the active agent(s) are useful for administering the active agent(s) to a subject. Any lipid vesicle suitable for encapsulating an antioxidant and for administering to the skin of a human subject may be used.

Vesicles of the invention are vesicles having one or more lipid bilayer membranes surrounding a cavity. Lipid vesicles for use in the invention are typically in the range of about 50 to about 950 nm (e.g., 50, 100, 200, 300, 400, 500, 600, 700, 800, 900, 950 nm) in size. Methods for producing and using lipid vesicles are well known in the art and are described, e.g., in U.S. Pat. Nos. 4,917,951 and 5,013,497; Walde P. and Ichikawa S., Biomol Eng., 18:143-177, 2001; Hunter D. G. and Frisken B. J., Biophys J., 74:2996-3002, 1998; and Cevc G., Adv Drug Deliv Rev., 56:675-711, 2004.

The antioxidant to be encapsulated within lipid vesicles can be any suitable form.

The compositions of the subject invention may comprise vesicles that contain only one active agent, or multiple active agents.

The lipid vesicles of the invention can include non-phospholipid surfactants. They can also include a charge-producing agent and a targeting molecule. Thus, vesicles made of non-phospholipid “membrane mimetic” amphiphiles are useful in the invention. These are molecules that have a hydrophilic head group attached to a hydrophobic tail and include long-chain fatty acids, long-chain alcohols and derivatives, long-chain amino and glycerolipids. In the bilayers, the fatty acid tails point into the membrane's interior and the polar head groups point outward. The polar groups at one surface of the membrane point towards the vesicle's interior and those at the other surface point toward the external environment. As a vesicle forms during its manufacture, any water-soluble molecules that have been added to the water are incorporated into the aqueous spaces in between the multiple layers of the lipid bilayer membrane, whereas any lipid-soluble molecules added during vesicle formation are incorporated into the core of the vesicles.

Paucilamellar vesicles that can be formed from many bio-compatible, single-tailed amphiphiles are preferred for use in the invention. Such paucilamellar lipid vesicles include non-phospholipid vesicles having one or several lipid bilayer membranes surrounding a large amorphous core in which a chemical entity of interest (i.e., an antioxidant) is encapsulated.

Non-phospholipid paucilamellar lipid vesicles are sold under the trade name Novasome® (IGI Inc., Buena, N.J.). Several Novasome® formulations exist (e.g., Novasome® A, Novasome® D, Novasome® Day Cream).

Novasome® vesicles are useful for encapsulating chemical ingredients to aid in formulation, increase delivery to site of action and stabilize chemical ingredients in the formulation. These lipid vesicles are generally about 200-700 nanometers in size, depending upon a wide variety of membrane constituents individually chosen for each particular purpose. Their size distribution is nearly uniform, and encapsulation efficiency can be nearly 100% for lipid cargo and 85% for aqueous materials. Finely divided insoluble particles (e.g., insoluble pharmaceuticals) can also be encapsulated.

Novasome® vesicles are inherently stable, and can be tailored to be stable at pH levels ranging from 2-13 as well as temperature ranges as low as liquid nitrogen to above the boiling point of water. They can be stable to solvents including alcohols, ethers, esters, gasoline, diesel and other fuels. They can encapsulate fragrances and flavors which contain volatile and fragile ethers, esters, aldehydes, etc. These vesicles can release their cargo under varying physical and chemical circumstances including heat, light, pH changes, enzymatic degradation, drying transmembrane diffusion, etc.

Protocols for producing and administering Novasome® formulations are described, for example, in U.S. Pat. Nos. 4,855,090; 4,911,928; 5,474,848; 5,628,936; 6,387,373; Holick et al., British Journal of Dermatology 149:370-376, 2003; Gupta et al., Vaccine 14:219-225, 1996; and Wallach D F H and Philippot J., New Type of Lipid Vesicle: Novasome™ In: Liposome Technology, 2^{nd} ed., Gregorriadis G., CRC Press, Boca Raton, Fla., 1982, pp. 141-151; Niemiec et al., Pharmaceutical Research 12:1184-1188, 1995; and Alfieri D R, Cosmetic Dermatology 10:42-52, 1997.

In one embodiment, the liposomes are those used in “Day Cream.”

In certain embodiments of the subject invention, the lipid vesicles (e.g., non-phospholipid paucilamellar lipid vesicles) may also include targeting molecules, either hydrophilic or amphiphilic, which can be used to direct the vesicles to a particular target in the skin in order to allow release of the antioxidant(s) from within the vesicle at a specified biological location. If hydrophilic targeting molecules are used, they can be coupled directly or via a spacer to an OH residue of the polyoxyethylene portion of the surfactant, or they can be coupled, using techniques in the art, to molecules such as palmitic acid, long chain amines, or phosphatidyl ethanolamine. If spacers are used, the targeting molecules can be interdigitated into the hydrophilic core of the bilayer membrane via the acyl chains of these compounds. Preferred hydrophilic targeting molecules include monoclonal antibodies, other immunoglobulins, lectins, and peptide hormones.

In addition to hydrophilic targeting molecules, it is also possible to use amphiphilic targeting molecules. Amphiphilic targeting molecules are normally not chemically coupled to the surfactant molecules but rather interact with the lipophilic or hydrophobic portions of the molecules constituting the bilayer lamellae of the lipid vesicles. Preferred amphiphilic targeting molecules are neutral glycolipids, galactocerebrosides (e.g., for hepatic galactosyl receptors), or charged glycolipids such as gangliosides.

In some embodiments, charge-producing materials and steroids such as cholesterol or hydrocortisone or their analogues and derivatives are used in the formation of the lipid vesicles (e.g., paucilamellar lipid vesicles). Preferred charge-producing materials include negative charge-producing materials such as dicetyl phosphate, cetyl sulphate, phosphatidic acid, phosphatidyl serine, oleic acid, palmitic acid, or mixtures thereof. In order to provide a net positive charge to the vesicles, long chain amines, e.g., stearyl amines or oleyl amines, long chain pyridinium compounds, e.g., cetyl pyridinium chloride, quaternary ammonium compounds, or mixtures of these can be used. Another example of a positive charge-producing material is hexadecyl trimethylammonium bromide, a potent disinfectant.

### Preparing Lipid Vesicles

Lipid vesicles used according to the subject invention can be any of a large variety of lipid vesicles known in the art and can be made according to any of a large number of production methods. Materials and procedures for forming lipid vesicles are well-known to those skilled in the art. In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large multilamellar vesicles are produced upon agitation. When smaller multilamellar vesicles are desired, the larger vesicles are subjected to sonication, sequential filtration through filters with decreasing pore size or reduced by other forms of mechanical shearing. Lipid vesicles can also take the form of unilamellar vesicles, which are prepared by more extensive sonication of multilamellar vesicles, and consist of a single spherical lipid bilayer surrounding an aqueous solution. A comprehensive review of all the aforementioned lipid vesicles and methods for their preparation are described in “Liposome Technology”, ed. G. Gregoriadis, CRC Press Inc., Boca Raton, Fla., Vol. I, II & III (1984). For methods of preparing lipid vesicles, also see U.S. Pat. Nos. 4,485,054, 4,761,288, 5,013,497, 5,653,996, and 6.855, 296.

To prepare non-phospholipid paucilamellar lipid vesicles formed of non-phospholipid surfactant material and containing an antioxidant, any suitable method known in the art can be used. Methods of preparing non-phospholipid paucilamellar lipid vesicles typically involve first forming a lipophilic phase by combining several lipophilic components including surfactant material and then heating and blending this mixture. Examples of suitable surfactant materials include but are not limited to polyoxyethylene (2) cetyl ether, polyoxyethylene (4) lauryl ether, glyceryl monostearate, and poly oxyethylene (9) glyceryl stearate. The resultant lipophilic phase is then blended with an aqueous phase having an aqueous buffer and an aqueous soluble collagen formulation, under shear mixing conditions, to form the paucilamellar lipid vesicles. In this method, the temperature of the lipophilic phase is elevated in order to make it flowable followed by carrying out the shear mixing between the lipophilic phase and the aqueous phase at a temperature such that both phases are liquids. While it is often desirable to use the same temperature for both phases, this is not always necessary. Any other method known to the skilled artisan can also be used. Preferred methods for making the paucilamellar lipid vesicles of the invention are described in U.S. Pat. No. 4,911,928.

To encapsulate oil-based antioxidants or antioxidant-containing formulations within paucilamellar lipid vesicles, the antioxidant or antioxidant-containing formulation is dispersed in an oil or wax forming an oily phase. The oil or wax is a water immiscible oily solution selected from a group consisting of oils, waxes, natural and synthetic triglycerides, acyl esters, and petroleum derivatives, and their analogues and derivatives. The oily phase containing the oil-dispersible material is mixed with the lipid phase and the combined oil-lipid phase is blended under shear mixing conditions with the aqueous phase. Surfactants useful in the encapsulation process are the same as those used to make paucilamellar lipid vesicles with an aqueous core.

Paucilamellar lipid vesicles can be made by a variety of devices which provide sufficiently high shear for shear mixing. Many such devices are available on the market including a Microfluidizer® such as is made by MicroFluidics Corp. (Newton, Mass.), a “French”-type press, or some other device which provides a high enough shear force and the ability to handle heated, semiviscous lipids. If a very high shear device is used, it may be possible to microemulsify powdered lipids, under pressure, at a temperature below their normal melting points and still form the antioxidant-containing paucilamellar lipid vesicles of the present invention.

A device which is particularly useful for making the paucilamellar lipid vesicles of the present invention has been developed by Micro Vesicular Systems, Inc., (Vineland, N.J.) and is further described in U.S. Pat. No. 4,895,452. Briefly, this device has a substantially cylindrical mixing chamber with at least one tangentially located inlet orifice. One or more orifices lead to a reservoir for the lipophilic phase, mixed with an oil phase if lipid-core paucilamellar lipid vesicles are to be formed, and at least one of the other orifices is attached to a reservoir for the aqueous phase. The different phases are driven into the cylindrical chamber through pumps, e.g., positive displacement pumps, and intersect in such a manner as to form a turbulent flow within the chamber. The paucilamellar lipid vesicles form rapidly, e.g., less than 1 second, and are removed from the chamber through an axially located discharge orifice. Preferably, there are four tangentially located inlet orifices and the lipid and aqueous phases are drawn from reservoirs, through positive displacement pumps, to alternating orifices. The fluid stream through the tangential orifices is guided in a spiral flow path from each inlet or injection orifice to the discharge orifice. The flow paths are controlled by the orientation or placement of the inlet or injection orifices so as to create a mixing zone by the intersection of the streams of liquid. The pump speeds, as well as the orifice and feed line diameters, are selected to achieve proper shear mixing for lipid vesicle formation. In most circumstances, turbulent flow is selected to provide adequate mixing.

No matter what device is used to form the paucilamellar lipid vesicles, if proper shear mixing is achieved they have a structure involving a large, unstructured amorphous center surrounded by a plurality of lipid bilayers having aqueous layers interspersed there between. About four lipid bilayers is standard with 2-8 possible. The amorphous center may be entirely filled with an aqueous material, e.g., a buffer and any aqueous material to be encapsulated, or may be partially or totally filled with an oily material, forming lipid-core paucilamellar lipid vesicles. If an aqueous center is used, the paucilamellar lipid vesicles will normally range in diameter from about 0.5-2μ while if an oily center is used, the size may increase to up to about 15-20μ depending upon the amount of oil used.

### Use of Cyclodextin as a Carrier

Additionally, cyclodextrins are an alternate option for an antioxidant carrier system into the dermis of the skin. Cyclodextrins are complex carbohydrates of 6, 7, or 8 D-glucopyranose residues that are linked by 1,4 glycosidic bonds. The three forms are dependent on the number of D-glucopyranose residues, the alpha form having 6, beta having 7, and gamma having 8. The alpha structure forms an annular ring with an internal hydrophobic cavity and a hydrophilic outer surface. Each cyclodextrin associates with a guest compound by fitting the compound into the hydrophobic cavity forming an inclusion complex. In this way cyclodextrins can be used as a delivery system to deliver a desired amount of material to a target location.

In one embodiment hydroxypropyl beta cyclodextrins can be used. Cyclodextrins are used because they have the ability to alter the physical, chemical, and biological properties of an associated guest compound through formation of the inclusion complex. This complex enhances the solubility, stability, and bioavailability of the guest compound so that the material can be isolated and used in a controlled delivery system. Formation of an inclusion complex of an antioxidant with an alpha-cyclodextrin allows for a targeted delivery system to the dermis.

The principal method for the isolation and purification of alpha-cyclodextrin takes advantage of its complex-forming ability. At completion of the reaction, 1-decanol is added to the reaction mixture to form an insoluble 1:1 alpha-cyclodextrin: 1-decanol inclusion complex. The complex is continuously mixed with water and separated from the reaction mixture by centrifugation. The recovered complex is re-suspended in water and dissolved by heating. Subsequent cooling leads to re-precipitation of the complex. The precipitate is recovered by centrifugation, and 1-decanol is removed by steam distillation. Upon cooling, alpha-cyclodextrin crystallizes from solution. The crystals are removed by filtration and dried, yielding a white crystalline powder with a water content under 11%. The purity on a dried basis is at least 98%.

### Dermatologically-Acceptable Carrier

The topical compositions of the present invention, in addition to the vesicle-contained antioxidant(s), can further comprise a dermatologically acceptable carrier. A safe and effective amount of carrier is typically from about 50% to about 99.99%, preferably from about 80% to about 99.9%, more preferably from about 90% to about 98%, and even more preferably from about 90% to about 95% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein.

Emulsions according to the present invention can contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973 to Dickert et al.; U.S. Pat. No. 4,421,769, issued Dec. 20, 1983 to Dixon et al.; and McCutcheon's Detergents and Emulsifiers, North American Ed., pages 317-324 (1986).

The emulsion may also contain an anti-foaming agent to minimize foaming upon application to the epidermal tissue. Anti-foaming agents include high molecular weight silicones and other materials well known in the art for such use.

Suitable emulsions may have a wide range of viscosities, depending on the desired product form. Exemplary low viscosity emulsions, which are preferred, have a viscosity of about 50 centistokes or less, more preferably about 10 centistokes or less, still more preferably about 5 centistokes or less.

Water-in-silicone emulsions can contain a continuous silicone phase and a dispersed aqueous phase. The continuous silicone phase exists as an external phase that contains or surrounds the discontinuous aqueous phase described hereinafter. The continuous silicone phase may contain one or more non-silicone oils. Examples of non-silicone oils suitable for use in the continuous silicone phase are those well known in the chemical arts in topical personal care products in the form of water-in-oil emulsions, e.g., mineral oil, vegetable oils, synthetic oils, semisynthetic oils, etc.

In emulsion technology, the term “dispersed phase” is a term well-known to one skilled in the art that means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore.

The aqueous phase can be water, or a combination of water and one or more water soluble or dispersible ingredients. Nonlimiting examples of such ingredients include thickeners, acids, bases, salts, chelants, gums, water-soluble or dispersible alcohols and polyols, buffers, preservatives, sunscreening agents, colorings, and the like.

Water-in-silicone emulsions can contain an emulsifier. In one embodiment, the composition contains from about 0.1% to about 10% emulsifier, more preferably from about 0.5% to about 7.5%, still more preferably from about 1% to about 5%, emulsifier by weight of the composition. The emulsifier helps disperse and suspend the aqueous phase within the continuous silicone phase.

Other topical carriers include oil-in-water emulsions, having a continuous aqueous phase and a hydrophobic, water-insoluble phase (“oil phase”) dispersed therein. Examples of suitable oil-in-water emulsion carriers are described in U.S. Pat. No. 5,073,371, to Turner, D. J. et al., issued Dec. 17, 1991, and U.S. Pat. No. 5,073,372, to Turner, D. J. et al., issued Dec. 17, 1991.

An oil-in-water emulsion can contain a structuring agent to assist in the formation of a liquid crystalline gel network structure. Structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

In certain embodiments, oil-in-water emulsions that contain at least one hydrophilic surfactant which can disperse the hydrophobic materials in the water phase (percentages by weight of the topical carrier). The surfactant, at a minimum, must be hydrophilic enough to disperse in water. Among the nonionic surfactants that are useful herein are those that can be broadly defined as condensation products of long chain alcohols, e.g. C8-30 alcohols, with sugar or starch polymers, i.e., glycosides.

Other suitable surfactants useful herein include a wide variety of cationic, anionic, zwitterionic, and amphoteric surfactants such as are known in the art. See, e.g., McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973; these four references are incorporated herein by reference in their entirety. The hydrophilic surfactants useful herein can contain a single surfactant, or any combination of suitable surfactants. The exact surfactant (or surfactants) chosen will depend upon the pH of the composition and the other components present.

Also useful herein are cationic surfactants, such as dialkyl quaternary ammonium compounds, examples of which are described in U.S. Pat. Nos. 5,151,209; 5,151,210; 5,120,532; 4,387,090; 3,155,591; 3,929,678; 3,959,461; McCutcheon's, Detergents & Emulsifiers, (North American edition 1979) M.C. Publishing Co.; and Schwartz, et al., Surface Active Agents, Their Chemistry and Technology, New York: Interscience Publishers, 1949; which descriptions are incorporated herein by reference.

A wide variety of anionic surfactants are also useful herein. See, e.g., U.S. Pat. No. 3,929,678, to Laughlin et al., issued Dec. 30, 1975, which is incorporated herein by reference in its entirety. Nonlimiting examples of anionic surfactants include the alkoyl isethionates, and the alkyl and alkyl ether sulfates.

Examples of amphoteric and zwitterionic surfactants are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilizing group, e.g., carboxy, sulfonate, sulfate, phosphate, or phosphonate.

The topical compositions of the subject invention, including but not limited to lotions and creams, may contain a dermatologically acceptable emollient. Such compositions preferably contain from about 1% to about 50% of the emollient. As used herein, “emollient” refers to a material useful for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of materials suitable as an emollient. A preferred emollient is glycerin. Glycerin is preferably used in an amount of from or about 0.001 to or about 30%, more preferably from or about 0.01 to or about 20%, still more preferably from or about 0.1 to or about 10%, e.g., 5%.

Creams are generally thicker than lotions due to higher levels of emollients or higher levels of thickeners.

Ointments of the present invention may contain a simple carrier base of animal or vegetable oils or semi-solid hydrocarbons (oleaginous); absorption ointment bases which absorb water to form emulsions; or water soluble carriers, e.g., a water soluble solution carrier. Ointments may further contain a thickening agent, such as described in Sagarin, Cosmetics, Science and Technology, 2nd Edition, Vol. 1, pp. 72-73 (1972), incorporated herein by reference, and/or an emollient. For example, an ointment may contain from about 2% to about 10% of an emollient; from about 0.1% to about 2% of a thickening agent; and the vesicle-collagen in the above described amounts.

### Additional Skin Care Agents

The compositions of the present invention may contain one or more additional skin care agents, in addition to one or more antioxidants, the agents enumerated below do not include water unless specifically stated.

The additional agents should be suitable for application to epidermal tissue, that is, when incorporated into the composition they are suitable for use in contact with human epidermal tissue without undue toxicity, incompatibility, instability, allergic response, and the like. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention.

Examples of such ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

In any embodiment of the present invention, however, the agents useful herein can be categorized by the benefit they provide or by their postulated mode of action. However, it is to be understood that the additional agents for use herein can in some instances provide more than one benefit or operate via more than one mode of action. Therefore, classifications herein are made for the sake of convenience and are not intended to limit the agent to that particular application or applications listed.

### Desquamation Agents

A safe and effective amount of a desquamation agent may be added to the compositions of the present invention, more preferably from about 0.1% to about 10%, even more preferably from about 0.2% to about 5%, also preferably from about 0.5% to about 4%, by weight of the composition. Desquamation agents enhance the skin appearance benefits of the present invention. For example, the desquamation agents tend to improve the texture of the skin (e.g., smoothness). One desquamation system that is suitable for use herein contains sulfhydryl compounds and zwitterionic surfactants and is described in U.S. Pat. No. 5,681,852, to Bissett, incorporated herein by reference. Another desquamation system that is suitable for use herein contains salicylic acid and zwitterionic surfactants and is described in U.S. Pat. No. 5,652,228 to Bissett, incorporated herein by reference. Zwitterionic surfactants such as described in these applications are also useful as desquamatory agents herein, with cetyl betaine being particularly preferred.

### Anti-Acne Agents

The compositions of the present invention may contain a safe and effective amount of one or more anti-acne agents. Examples of useful anti-acne agents include resorcinol, sulfur, salicylic acid, benzoyl peroxide, erythromycin, zinc, etc. Further examples of suitable anti-acne agents are described in further detail in U.S. Pat. No. 5,607,980, issued to McAtee et al, on Mar. 4, 1997.

### Wrinkle Agents/Anti-Atrophy Agents

The compositions of the present invention may further contain a safe and effective amount of one or more anti-wrinkle agents or anti-atrophy agents. Exemplary anti-wrinkle/anti-atrophy agents suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, skin peel agents (e.g., phenol and the like), vitamin B₃ compounds, retinoids, and hyaluronic acid, which enhance the epidermal tissue appearance benefits of the present invention, especially in regulating epidermal tissue condition, e.g., skin condition.

### Hyaluronic Acid

The compositions of the present invention may optionally contain hyaluronic acid (HA), which can be linear HA. The HA can be cross-linked or not cross-linked.

If the HA is to be cross-linked, numerous substances can be used to cross-link HA including formaldehyde, epoxides, polyaziridyl compounds, divinyl sulfone and others. One cross-linking agent is divinyl sulfone. This substance reacts readily with HA in aqueous alkaline solutions, thereby providing cross-linked HA gels. These gels swell in water. The swelling ratio depends upon the degree of cross-linking of the gel. The degree of cross-linking can be controlled by changing several factors including the molecular weight of the HA, its concentration in the reaction mixture, the alkali concentration and the polymer/DVS ratio. The swelling ratio of these gels can be from 20 up to 8000, and more, depending upon the reaction parameters. Another cross-linking agent is 1,4-butanediol diglycidyl ether (BDDE).

The HA may also optionally be in the form of a monophasic gel. Additionally, HA can be used to deliver other active agents by covalently attaching such an active agent to the HA to form a gel.

### Collagen

The compositions of the present invention may optionally include one or more purified, or recombinant, collagens and/or collage derivatives, or a combination thereof. Collagen proteins useful in the invention include any native collagen proteins obtained from animal (e.g., human) cells and tissue, recombinantly expressed human collagen proteins (including fragments of the full-length collagen), and combinations and/or formulations thereof.

Purified collagens for use in the methods and compositions of the invention may be isolated from animal or human tissues; however, the use of human collagen in the compositions and methods of the invention is preferred when the subject to be treated is a human in order to prevent an immune response to the collagen material. Collagen that is extracted from its source material (e.g., animal placenta, bone, hide, tendon) is typically a mixture of collagen type I with some collagen type III. Collagen material recovered from placenta, for example, is biased as to collagen type and not entirely homogenous. Techniques for isolating collagen from human placentas are described in U.S. Pat. Nos. 5,002,071 and 5,428,022.

In addition to employing collagen obtained directly from natural sources, the methods and compositions of the invention include many different types of collagen derivatives. Collagen derivatives may vary from naturally-occurring collagens in several respects. Collagen derivatives may be non-glycosylated or glycosylated differently than naturally-occurring collagens. Desired glycosylation patterns may be produced by a variety of methods, including direct chemical modification and enzymatically catalyzed glycosylation and deglycosylation reactions. Desired glycosylation patterns may also be produced by inhibiting or deleting enzymes necessary for producing the naturally-occurring glycosylation patterns found on collagens.

Collagen derivatives also include various fragments of naturally-occurring collagens. Such collagen fragments may be produced by, among other methods, chemically or enzymatically cleaving one or more peptide bonds. Collagen derivatives may also contain one or more amino acid residue differences as compared with corresponding amino acid residue positions in a naturally-occurring collagen. Collagen derivatives containing such amino acid residue substitutions may be produced by a variety of methods including genetic engineering techniques and by in vitro peptide synthesis. Additional collagen derivatives may be produced by varying the amount of hydroxylysines and/or hydroxyprolines present in a given molecule, by the varied expression of lysine hydroxylases, and/or proline hydroxylases, wherein the hydroxylase genes (recombinant or otherwise) are also expressed in a host cell for the expression of recombinant collagen, or derivatives thereof.

Regardless of the collagen source, preferred collagen and collagen derivatives for use in the invention arc those that are sized to fit within the lipid vesicles of the invention, e.g., less than about 800 nm. Because collagen fibrils are 20-150 nm in size, fibrils rather than fibers (which are 1000-50,000 nm) are preferred. To maintain collagen in the fibril form, the pH and/or ionic strength of the solution containing the fibrils can be appropriately manipulated. A number of methods exist to reduce collagen size, including an enzymatic breakdown using a protease. Collagen can also be broken down mechanically. For example, collagen can be processed mechanically after drying to produce fine particles that are less than 800 nm in size. Additionally, extensive hydrolysis of a collagen-containing solution may be used to prevent fiber formation.

Vitamin B₃ Compounds

The compositions of the present invention may contain a safe and effective amount of a vitamin B₃ compound. Vitamin B₃ compounds are particularly useful for regulating skin condition as described in U.S. application Ser. No. 08/834,010, filed Apr. 11, 1997 (corresponding to international publication WO 97/39733 A1, published Oct. 30, 1997). Examples of suitable vitamin B₃ compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.). The vitamin compounds may be included as the substantially pure material, or as an extract obtained by suitable physical and/or chemical isolation from natural (e.g., plant) sources.

### Retinoids

The compositions of the present invention may also contain a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), more preferably retinoids other than retinoic acid. These compounds are well known in the art and are commercially available from a number of sources, e.g., Sigma Chemical Company (St. Louis, Mo.), and Boerhinger Mannheim (Indianapolis, Ind.). Other retinoids which are useful herein are described in U.S. Pat. No. 4,677,120, issued Jun. 30, 1987 to Parish et al.; U.S. Pat. No. 4,885,311, issued Dec. 5, 1989 to Parish et al.; U.S. Pat. No. 5,049,584, issued Sep. 17, 1991 to Purcell et al.; U.S. Pat. No. 5,124,356, issued Jun. 23, 1992 to Purcell et al.; and U.S. Pat. No. Reissue 34,075, issued Sep. 22, 1992 to Purcell et al. Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6[2-(4,4-dimethylthiochroman-6-yl)-ethynyl]nicotinate). Preferred retinoids are retinol, retinyl palmitate, retinyl acetate, retinyl propionate, retinal and combinations thereof.

### Hydroxy Acids

The compositions of the present invention may contain a safe and effective amount of a hydroxy acid. Preferred hydroxy acids for use in the compositions of the present invention include salicylic acid and salicylic acid derivatives.

### Chelators

The compositions of the present invention may also contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. Preferred chelators useful in compositions of the subject invention are furildioxime, furilmonoxime, and derivatives thereof.

### Flavonoids

The compositions of the present invention may optionally contain a flavonoid compound. Flavonoids are broadly disclosed in U.S. Pat. Nos. 5,686,082 and 5,686,367, both of which are herein incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, 0-glycoside, and the like or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

Preferred for use herein are unsubstituted flavanone, methoxy flavanones, unsubstituted chalcone, 2′,4-dihydroxy chalcone, and mixtures thereof. More preferred are unsubstituted flavanone, unsubstituted chalcone (especially the trans isomer), and mixtures thereof.

They can be synthetic materials or obtained as extracts from natural sources (e.g., plants). The naturally sourced material can also further be derivatized (e.g., an ester or ether derivative prepared following extraction from a natural source). Flavonoid compounds useful herein are commercially available from a number of sources, e.g., Indofine Chemical Company, Inc. (Somerville, N.J.), Steraloids, Inc. (Wilton, N.H.), and Aldrich Chemical Company, Inc. (Milwaukee, Wis.).

### Anti-Cellulite Agents

The compositions of the present invention may also contain a safe and effective amount of an anti-cellulite agent. Suitable agents may include, but are not limited to, xanthine compounds (e.g., caffeine, theophylline, theobromine, and aminophylline).

### Topical Anesthetics

The compositions of the present invention may also contain a safe and effective amount of a topical anesthetic. Examples of topical anesthetic drugs include articaine, benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

### Tanning Agents

The compositions of the present invention may contain a tanning agent. When present, it is preferable that the compositions contain from about 0.1% to about 20%, more preferably from about 2% to about 7%, and still more preferably from about 3% to about 6%, by weight of the composition, of dihydroxyacetone as an artificial tanning agent.

### Skin Lightening Agents

The compositions of the present invention may contain a skin lightening agent. When used, the compositions preferably contain from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in the PCT publication No. 95/34280, in the name of Hillebrand, corresponding to PCT Application No. U.S. 95/07432, filed Jun. 12, 1995; and co-pending U.S. application Ser. No. 08/390,152 filed in the names of Kvalnes, Mitchell A. DeLong, Barton J. Bradbury, Curtis B. Motley, and John D. Carter, corresponding to PCT Publication No. 95/23780, published Sep. 8, 1995.

### Skin Soothing and Skin Healing Agents

The compositions of the present invention may comprise a skin soothing or skin healing agent. Skin soothing or skin healing agents suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing agent may be added to the present composition, preferably, from about 0.1% to about 30%, more preferably from about 0.5% to about 20%, still more preferably from about 0.5% to about 10%, by weight of the composition formed.

### Antimicrobial and Antifungal Agents

The compositions of the present invention may contain an antimicrobial or antifungal agent. Such agents are capable of destroying microbes, preventing the development of microbes or preventing the pathogenic action of microbes. A safe and effective amount of an antimicrobial or antifungal agent may be added to the present compositions, preferably, from about 0.001% to about 10%, more preferably from about 0.01% to about 5%, and still more preferably from about 0.05% to about 2%.

Examples of antimicrobial and antifungal agents include B-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Additionally, antimicrobial peptides can be used.

### Conditioning Agents

The compositions of the present invention may contain a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of fat Has (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose, glucosamine); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to On et al, issued Dec. 11, 1990.

### Structuring Agents

The compositions hereof, and especially the emulsions hereof, may contain a structuring agent. Structuring agents are particularly preferred in the oil-in-water emulsions of the present invention. Without being limited by theory, it is believed that the structuring agent assists in providing rheological characteristics to the composition which contribute to the stability of the composition. For example, the structuring agent tends to assist in the formation of the liquid crystalline gel network structures. The structuring agent may also function as an emulsifier or surfactant. Preferred compositions of this invention contain from about 0.1% to about 20%, more preferably from about 0.1% to about 10%, still more preferably from about 0.5% to about 9%, of one or more structuring agents.

The preferred structuring agents of the present invention are selected from stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from stearic acid, palmitic acid, stearyl alcohol, cetyl alcohol, behenyl alcohol, steareth-2, and mixtures thereof.

### Thickening Agent (Including Thickeners and Gelling Agents)

The compositions of the present invention can contain one or more thickening agents, preferably from about 0.1% to about 5%, more preferably from about 0.1% to about 4%, and still more preferably from about 0.25% to about 3%, by weight of the composition.

Nonlimiting classes of thickening agents for use in the compositions of the invention include those selected from the following: carboxylic acid polymers (such as those described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80); crosslinked polyacrylate polymers (such as those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987); polyacrylamide polymers (such as nonionic polyacrylamide polymers including substituted branched or unbranched polymers and multi-block copolymers of acrylamides and substituted acrylamides with acrylic acids and substituted acrylic acids); polysaccharides (which refers to gelling agents that contain a backbone of repeating sugar (i.e., carbohydrate) units, including cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); and gums (such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof).

### Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

### Preservatives

Preservatives can be incorporated into the compositions of the present invention to protect against the growth of potentially harmful microorganisms. While it is in the aqueous phase that microorganisms tend to grow, microorganisms can also reside in the anhydrous or oil phase. As such, preservatives, which have solubility in both water and oil, arc preferably employed in the present compositions. Suitable traditional preservatives for compositions of this invention are alkyl esters of parahydroxybenzoic acid. Other preservatives, which can be used include hydantoin derivatives, propionate salts, and a variety of quaternary ammonium compounds.

Particularly preferred preservatives are methylparaben, imidazolidinyl urea, sodium dehydroacetate, propylparaben, trisodium ethylenediamine tetraacetate (EDTA), and benzyl alcohol. The preservative can be selected to avoid possible incompatibilities between the preservative and other ingredients. Preservatives are preferably employed in amounts ranging from about 0.01% to about 2% by weight of the composition. Other preservatives known in the art can be used in the present invention.

### Methods of Administration

Another aspect of the invention is to provide a method of administering a composition of the invention, wherein dispersed lipid vesicles and/or cyclodextrins comprising one or more antioxidants are provided to the dermal layer of a patient's skin. The method includes the step of contacting the skin or other target site of the subject with a composition including a lipid vesicle (e.g., non-phospholipid paucilamellar lipid vesicle) having a cavity containing one or more antioxidants.

The compositions of the present invention are useful for regulating and/or improving mammalian skin condition. Such regulation of epidermal tissue conditions can include prophylactic and therapeutic regulation. For example, such regulating methods are directed to thickening dermal tissue and preventing and/or retarding atrophy of mammalian skin, preventing and/or retarding the appearance of spider vessels and/or red blotchiness on mammalian skin, preventing and/or retarding the appearance of dark circles under the eye of a mammal, preventing and/or retarding sallowness of mammalian skin, preventing and/or retarding sagging of mammalian skin, softening and/or smoothing lips of a mammal, preventing and/or relieving itch of mammalian skin, regulating skin texture (e.g. wrinkles and fine lines), and improving skin color (e.g. redness, freckles).

Regulating epidermal tissue condition involves topically applying to the epidermal tissue a safe and effective amount of a composition of the present invention. The amount of the composition which is applied, the frequency of application and the period of use will vary widely depending upon the level of antioxidant(s) (and, when present, other skin care agents) of a given composition and the level of regulation desired, e.g., in light of the level of epidermal tissue damage present or expected to occur.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic application continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions which are typically applied per application are, in mg composition/cm² skin, from about 0.1 mg/cm² to about 10 mg/cm². A particularly useful application amount is about 1 mg/cm² to about 2 mg/cm².

Improving and/or regulating epidermal tissue condition is preferably practiced by applying a composition in the form of a skin lotion, cream, gel, foam, ointment, paste, emulsion, spray, conditioner, tonic, cosmetic, lipstick, foundation, after-shave, or the like which is preferably intended to be left on the skin or other keratin structure for some esthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, still more preferably for at least several hours, e.g., up to about 12 hours. Any part of the external portion of the body can be treated, e.g., lips, under-eye area, eyelids, scalp, neck, torso, arms, hands, legs, feet, etc. The composition can be applied with the fingers or with an implement or device (e.g., pad, cotton ball, applicator pen, spray applicator, and the like).

Another approach to ensure a continuous dispersal of at least a minimum level of an antioxidant (and, when present, at least one skin care agent) to the dermal layer is to apply the compound by use of a patch applied, e.g., to the face. Such an approach is particularly useful for problem skin areas needing more intensive treatment (e.g., facial crows feet area, frown lines, under eye area, and the like). The patch can be occlusive, semi-occlusive or non-occlusive and can be adhesive or non-adhesive. The composition can be contained within the patch or be applied to the skin prior to application of the patch. The patch can also include additional agents such as chemical initiators for exothermic reactions such as those described in U.S. Pat. Nos. 5,821,250, 5,981,547, and 5,972,957 to Wu, et al. The patch is preferably left on the skin for a period of at least about 5 minutes, more preferably at least about 15 minutes, more preferably still at least about 30 minutes, even more preferably at least about 1 hour, still more preferably at night as a form of night therapy.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A liposome comprising at least one ingredient selected from the group consisting of antioxidants, anti-inflammatory agents, peptides, humectants, sunscreen agents, and emollients.

2. The liposome, according to claim 1, comprising one or more of the following antioxidants: L-ascorbic acid, Vitamin E (tocopherol), tocopheryl acetate, Coenzyme Q-10, white tea extract, grape seed extract, niacinamide, and zinc citrate.

3. The liposome, according to claim 1, which is paucilamellar.

4. A method for administering an agent into the skin of a subject, the method comprising the step of contacting the skin of the subject with a composition comprising a carrier comprising a cavity having encapsulated therein at least one ingredient selected from the group consisting of antioxidants, anti-inflammatory agents, peptides, humectants, sunscreen agents, and emollients, wherein said carrier is a liposome or a cyclodextrin.

5. The method, according to claim 4, wherein the antioxidant is one of the following: Vitamin E (tocopherol), tocopheryl acetate, Coenzyme Q-10, white tea extract, grape seed extract, niacinamide, and zinc citrate.

6. The method, according to claim 4, wherein said carrier is a liposome.

7. The method, according to claim 6, wherein the liposome is paucilamellar.

8. (canceled)

9. The liposome, according to claim 1, comprising one or more of the following anti-inflammatory agents: cucumber extract, ivy extract, shitake extract, and/or allantoin.

10. (canceled)

11. The method according to claim 4, comprising the step of contacting the skin of the subject with a composition comprising a carrier comprising a cavity having encapsulated therein an anti-inflammatory agent.

12. The method, according to claim 11, wherein the anti-inflammatory agent is one of the following: cucumber extract, ivy extract, shitake extract, and/or allantoin.

13-15. (canceled)

16. The liposome, according to claim 1, comprising one or more of the following peptides: hexapeptide-3 (Argireline), hexapeptide-9 (Collaxyl), Dermaxyl™ (palmitoyl oligopeptide), Matrixyl 3000™ (glycerin, butylene glycol, water, carbomer, polysorbate-20, palmitoyl oligopeptide, and palmitoyl tetrapeptide-3), Haloxyl™ (palmitoyl tetrapeptide-3), Sepilift™ (dipalmitoyl hydroxyproline), Eyeliss™ (hesperidin methyl chalcone and dipeptide-2 with palmitoyl tetrapeptide-3), Rigin, and/or Maxilip™ (ethylhexyl palmitate, tribehenin, sorbitan isostearate, and palmitoyl oligopeptide).

17. (canceled)

18. The method according to claim 4, comprising the step of contacting the skin of the subject with a composition comprising a carrier comprising a cavity having encapsulated therein a peptide.

19. The method, according to claim 18, wherein the peptide is one of the following: hexapeptide-3 (Argireline), hexapeptide-9 (Collaxyl), Dermaxyl™ (palmitoyl oligopeptide), Matrixyl 3000™ (glycerin, butylene glycol, water, carbomer, polysorbate-20, palmitoyl oligopeptide, and palmitoyl tetrapeptide-3), Haloxyl™ (palmitoyl tetrapeptide-3), Sepilift™ (dipalmitoyl hydroxyproline), Eyeliss™ (hesperidin methyl chalcone and dipeptide-2 with palmitoyl tetrapeptide-3), Rigin, and/or Maxilip™ (ethylhexyl palmitate, tribehenin, sorbitan isostearate, and palmitoyl oligopeptide).

20-22. (canceled)

23. The liposome, according to claim 1, comprising one or more of the following humectants: avocado oil/sterol, avocado butter, white petrolatum, and/or illipe butter.

24. (canceled)

25. The method according to claim 4, comprising the step of contacting the skin of the subject with a composition comprising a carrier comprising a cavity having encapsulated therein a humectant.

26. The method, according to claim 25, wherein the humectant is one of the following: avocado oil/sterol, avocado butter, white petrolatum, and/or illipe butter.

27-29. (canceled)

30. The liposome, according to claim 1, comprising one or more of the following sunscreen agents: octocrylene, zinc oxide, and/or octyl methoxycinnannate.

31. (canceled)

32. The method according to claim 4, comprising the step of contacting the skin of the subject with a composition comprising a carrier comprising a cavity having encapsulated therein a sunscreen agent.

33. The method, according to claim 32, wherein the sunscreen agent is one of the following: octocrylene, zinc oxide, octyl methoxycinnannate.

34-36. (canceled)

37. The liposome, according to claim 1, comprising one or more of the following emollients: illipe butter, shea butter, shora seed butter, Ceraphyl 847® (octyldodecyl stearoyl stearate), C12-15 alkyl benzoate, pentaerythrityl tetraisostearate, and/or diisopropyl adipate.

38. (canceled)

39. The method according to claim 4, comprising the step of contacting the skin of the subject with a composition comprising a carrier comprising a cavity having encapsulated therein an emollient.

40. The method, according to claim 39, wherein the emollient is one of the following: illipe butter, shea butter, shora seed butter, Ceraphyl 847® (octyldodecyl stearoyl stearate), C12-15 alkyl benzoate, pentaerythrityl tetraisostearate, and/or diisopropyl adipate.

41-42. (canceled)
